(19) United States
(12) Patent Application Publication
(10) Pub. No.: US 2023/0415259 A1
(43) Pub. Date: Dec. 28, 2023

(54) MATERIAL PROCESSING APPARATUS AND METHOD FOR PREPARING A MATERIAL PROCESSING APPARATUS

(71) Applicant: SCHWIND eye-tech-solutions GmbH, Kleinostheim (DE)

(72) Inventors: Samuel ARBA MOSQUERA, Aschaffenburg (DE); Nico TRIEFENBACH, Mainaschaff (DE)

(21) Appl. No.: 18/331,573

(22) Filed: Jun. 8, 2023

(30) Foreign Application Priority Data
Jun. 22, 2022 (DE) 10 2022 115 566.8

(51) Int. Cl. B23K 26/06; B23K 26/082

(52) U.S. Cl. CPC B23K 26/0643; B23K 26/082

(57) ABSTRACT

The invention relates to a method for preparing a material processing apparatus (10) for material processing of an object, wherein a contact element (20) to be fitted onto the object is attached to the material processing apparatus (10), wherein the contact element (20) is transparent to processing laser radiation, comprises a contact surface (22) on its side to be fitted onto the object, and an entry surface (24) on its side facing the material processing apparatus, wherein a shape of the contact surface (22) and/or entry surface (24) is determined by means of radiation of measurement laser radiation (14) before processing the object, in that the measurement laser radiation (14) is focused on the contact surface (22) and/or the entry surface (24) by means of the variable focus adjusting device (18), wherein radiation backscattered or back-reflected from the focus of the measurement laser radiation (14) is confocally detected and thus a position of intersections on the contact surface (22) and/or entry surface (24) is determined, wherein a three-dimensional surface model is adapted to the determined position of the intersections, wherein the 3-dimensional shape of the contact surface (22) and/or entry surface (24) is provided by the surface model.

### FIELD

The invention relates to a method for preparing a material processing apparatus for material processing by generating optical breakthroughs in or on an object as well as to a material processing apparatus, which is formed to perform the method. Furthermore, the invention relates to a computer program including commands, which cause the material processing apparatus to perform the method, as well as to a computer-readable medium, on which the computer program is stored.

### BACKGROUND

In material processing, a laser is often focused on areas of an object to be processed, wherein processing laser radiation is employed hereto, the intensity of which is high enough to generate optical breakthroughs. In order that the processing laser radiation can be focused on previously determined positions, it is usually indispensable that the object is oriented and retained in an exactly defined position to the processing laser radiation. For retaining the object in the exactly defined position, a contact element is usually used, by which the object to be processed can be fixed in a position, whereby defined conditions are achievable. Thereby, the contact element becomes part of the optical path of the processing laser radiation.

This is in particular required in the microprocessing of materials, which only have a low linear optical absorption in the spectral range of the processing laser radiation, or in the generation of structures within the object, in particular in the laser-induced refractive index change (LIRIC). In such materials, non-linear interactions between laser radiation and material are usually exploited, mostly in the form of an optical breakthrough, which is generated in the focus of high-energy laser radiation. Since the processing effect then only occurs in the laser beam focus, it is important to exactly three-dimensionally orient the position of the focus. In addition to a two-dimensional deflection of the laser beam, an exact depth adjustment of the focus position is thus required. The contact element serves for ensuring constant optical conditions also known with a certain accuracy in the optical path to the object in that the object and the laser processing apparatus are mechanically coupled by the contact element and a shape with known optical effect is additionally provided to the object surface.

A typical application for such a contact element is in ophthalmological methods, in particular in an ablation and/or photodisruption and/or laser-induced refractive index change (LIRIC), wherein the contact element, which can for example include glass, plastic, PMMA and/or polymers, is to transparently act at least to processing laser radiation. Therein, the material processing apparatus is formed with an ophthalmological laser, which focuses laser radiation into the cornea. An optical breakthrough can arise in the focus, which causes a local separation of the corneal tissue. By suitable succession of these optical breakthroughs, corneal layers can then be ablated or a corneal volume can be isolated and removed.

A shape and position of the contact element is accuracy-determining in such a material processing, wherein the position of the contact element to the material processing apparatus is determined after coupling to it and before the material processing within the scope of the preparation of the material processing apparatus. Therein, the basic shape of the contact element is usually known, wherein it can possibly slightly deviate from a specified shape.

From WO 2008/040436 A1, a generic apparatus and a method for preparing the apparatus for material processing by generating optical breakthroughs in or on an object are known. The apparatus comprises a variable, three-dimensionally acting focus adjusting device for focusing pulsed processing laser radiation on various locations in or on the object, wherein a contact element to be fitted onto the object is attached to the apparatus, wherein the contact element is transparent to the processing laser radiation and comprises a curved contact surface of previously known shape on its side to be fitted onto the object, wherein the position of the contact surface with respect to the focus adjusting device is determined by means of radiation of measurement laser radiation onto the contact surface before processing the object in that the measurement laser radiation is focused near or on the contact surface by means of the variable focus adjusting device. The energy density of the focused measurement laser radiation is too low for generating an optical breakthrough and the focus position of the measurement laser radiation in a measurement surface is adjusted such that it intersects the expected position of the contact surface, wherein radiation backscattered or back-reflected from the focus of the measurement laser radiation is confocally detected, wherein the position of intersections between measurement surface and contact surface is determined from the confocally detected radiation and the associated adjustment of the variable focus adjusting device, wherein the position of the contact surface is determined from the position of the intersections and the previously known shape of the contact surface.

It is disadvantageous in such a determination of the position of the contact element that the exact shape of the contact element has to be previously known to be able to calculate back the position of the contact surface from the intersections. However, contact elements can have certain tolerances, which can falsify such a position determination.

Therefore, it is the object of the present invention to improve a preparation of the material processing apparatus, in particular to avoid the disadvantages or the prior art.

### SUMMARY

This object is solved by the method according to the invention, the apparatuses according to the invention, the computer program according to the invention as well as the computer-readable medium according to the invention. Advantageous configurations with convenient developments of the invention are specified in the respective dependent claims, wherein advantageous configurations of the method are to be regarded as advantageous configurations of the treatment apparatus, of the control device, of the computer program as well as of the computer-readable medium and vice versa.

The invention is based on the idea that the shape of the contact element, in particular of a contact surface and/or entry surface, and thereby the position with respect to the processing laser radiation can directly be determined by means of measurement laser radiation without previously knowing the exact shape thereto. Hereto, a plurality of intersections of the laser radiation with the contact surface and/or entry surface can be determined, from which the shape can then be calculated.

By the invention, a method for preparing a material processing apparatus for material processing by generating optical breakthroughs in or on an object is provided. The material processing apparatus comprises a variable, three-dimensionally acting focus adjusting device for focusing processing laser radiation on various locations in or on the object, wherein a contact element to be fitted onto the object is attached to the material processing apparatus. The contact element is transparent to the processing laser radiation, comprises a contact surface on its side to be fitted onto the object, and comprises an entry surface for the processing laser radiation on its side facing the material processing apparatus. Before the processing of the object, a shape of the contact surface and/or entry surface is determined by means of radiation of measurement laser radiation onto the contact surface and/or entry surface in that the measurement laser radiation is focused near or on the contact surface and/or entry surface by means of the variable focus adjusting device, wherein an energy density of the focused measurement laser radiation is too low for generating an optical breakthrough, wherein radiation backscattered or back-reflected from the focus of the measurement laser radiation is confocally detected, wherein a position of intersections on the contact surface and/or entry surface is determined from the confocally detected radiation and the associated adjustment of the variable focus adjusting device. A three-dimensional surface model is adapted to the determined position of the intersections, wherein the 3-dimensional shape of the contact surface and/or entry surface is provided by the surface model.

In other words, the material processing apparatus can comprise one or more lasers, wherein the laser or lasers are formed for providing processing laser radiation, by which optical breakthroughs can arise in the object. In addition, the laser or lasers can be formed to provide measurement laser radiation, the energy of which is too low for generating optical breakthroughs. Preferably, the measurement laser radiation can be focused on the contact surface and/or entry surface of the contact element by the same focus adjusting device, which is used for focusing the processing laser radiation. The contact element, which is transparent to the processing laser radiation, can previously be coupled to the material processing apparatus such that it is in the optical path of the processing laser radiation and/or the measurement laser radiation.

In particular, a refractive index jump can occur at a transition from air to the contact surface and/or entry surface of the contact element, by which backscattered or back-reflected radiation can be distinguished compared to focusing in air and/or within the contact element. Thus, it is allowed, in particular by means of a confocal measurement, to determine intersections of the measurement laser radiation on the contact surface and/or entry surface. Therein, the confocal detection of the backscattered or reflected measurement laser radiation advantageously exploits that the portion of the transmission radiation backscattered at the interface of a transparent medium, which is confocally detected, is significantly higher than within the transparent contact element. By the spatial filtering occurring therein, the confocal detection provides a sufficient signal, the strength of which is substantially dependent on the refractive index difference of the media adjoining to each other on the contact surface. Herein, the principle of the confocal measurement is known from the prior art.

After the position of intersections on the contact surface and/or entry surface has been determined, a three-dimensional surface model can be adapted to the determined position of the intersections. This means that mathematical models can for example be fitted to the intersections to determine the shape of the contact surface and/or entry surface. Thus, it is not required to previously know the shape of the contact surface and/or entry surface to determine the position of the contact element with respect to the material processing apparatus. By the method, the shape of the respective surface can be directly determined in the coordinate system of the material processing apparatus and the processing laser radiation, respectively, whereby the position of the contact element is also known at the same time. Hereto, it is apparent for the expert that an improved determination of the three-dimensional shape of the contact surface and/or the entry surface can be achieved with increase of the number of the intersections. Therefore, for the application, the expert will search for a sufficient number of intersections and/or optionally repeat the irradiation of measurement laser radiation until a sufficiently high number of intersections is found. With knowledge of a basic shape of the contact element, a suitable distribution of the measurement laser radiation and thereby of the intersections can preferably also be selected to scan this shape.

Preferably, the processing laser radiation and/or measurement laser radiation can be pulsed laser radiation, wherein an energy range of the measurement laser radiation is below an energy for an optical breakthrough, in particular below one Joule per square centimeter or a power density per pulse below 10⁹ watts per square centimeter.

The advantage arises by the invention that a preparation for a material processing can be improved in that a previous knowledge of the contact element is not required since the shape and thus the position with respect to the material processing apparatus and the processing laser radiation, respectively, can thus be directly determined by means of the method. In particular, a radiation planning can then be adapted to the determined shape.

The invention also includes embodiments, by which additional advantages arise.

An embodiment provides that a grid structure is provided by the position of the intersections on the contact surface and/or the entry surface, wherein polygons are adapted to the grid structure as the three-dimensional surface model. This means that a space is scanned by the measurement laser radiation, in which the contact surface and/or the entry surface are located, wherein the confocally detected intersections with the respective surface are present as a three-dimensional grid structure. Then, polygons can be adapted to this grid structure, which result in the three-dimensional shape of the respective surface. For example, one obtains the polygons in that one connects the intersections, which form the grid structure, in particular adjacent intersections, to each other such that a closed surface arises between the grid points. This can preferably be performed for all of the grid points of the grid structure to obtain the shape of the respective surface with a closed polygonal line. Thus, the three-dimensional shape of the respective surface in the space can be determined in simple manner.

A further configuration provides that polynomials, in particular a polynomial line, are adapted to the intersections on the contact surface and/or the entry surface. In other words, a function, which is piecewise composed of n-th order polynomials, for example bicubic, can be adapted to the intersections. Such splines (polynomial line) can then be used to interpolate the intersections and the intermediate surface and thus to provide the three-dimensional shape of the contact surface and/or entry surface. Preferably, this can be performed by fit algorithms, which use the intersections in one or more planes to fit the polynomials thereto. Hereby, the advantage arises that a further preferred embodiment can be provided.

A further embodiment provides that Zernike polynomials or a Fourier series are adapted to the intersections on the contact surface and/or entry surface as the three-dimensional surface model. This means that Zernike polynomials can in particular be adapted to the intersections as polynomials, which provide smooth and derivable surfaces. In particular in the ophthalmology, Zernike polynomials are used for representation of wavefronts, wherein the determination of the three-dimensional shape of the contact surface and/or entry surface by Zernike polynomials is in particular suitable in case of contact elements for the ophthalmology. Alternatively, a Fourier series can be adapted to the intersections, wherein the Fourier series represents a periodic, piecewise continuous function of sine and cosine functions. By this embodiment, further suitable three-dimensional surface models for determining the three-dimensional shape of the contact surface and/or entry surface can be obtained.

A further embodiment provides that the measurement laser radiation is focused near or on the contact surface and/or entry surface according to a preset scan strategy. This means that a scan strategy can be provided to obtain sufficient intersections with the contact surface and/or the entry surface, to adapt the three-dimensional surface model thereto. According to used three-dimensional surface model, a different scan strategy can be used. Herein, a sufficient number of intersections can for example first be searched, wherein a measurement can be repeated upon falling below a threshold, in particular with changed scanning area, until sufficient intersections are achieved. In particular, the expert will determine the required number of intersections for the respectively used three-dimensional surface model from empirical values and/or experiments. The expert also adapts a desired accuracy in the determination of the three-dimensional shape and the intersections required thereto corresponding to the desired accuracy. For example, hexagonal patterns, rectangular patterns and/or circular patterns can be used as the preset scan strategy. Alternatively or additionally, an Albrecht distribution and/or a Jacobi distribution and/or a Legendre distribution can be used to scan a surface. Preferably, one of the above-mentioned patterns or distributions can be scanned in an x-y plane, wherein the z-position (depth direction) is subsequently adjusted and the next plane is again scanned with one of these scan strategies. This can be performed for multiple z-positions until a sufficiently high number of intersections is found.

Preferably, it is provided that focus points of the measurement laser radiation are uniformly distributed in a spatial area, in which the contact surface and/or entry surface are expected, according to the scan strategy. Thus, in the scan strategy, the measurement laser radiation can already be focused into those areas by the focus adjusting device, in which the contact surface and/or the entry surface are empirically expected. The focus points of the measurement laser radiation can then be uniformly distributed in this three-dimensional spatial area to confocally detect intersections.

Particularly preferably, it is provided that the focus adjusting device is adjusted for focusing the measurement laser radiation on an x-y-position, which is located in a surface situated perpendicularly to the radiation direction of the focus adjusting device, and multiple focus points are scanned along a z-axis, which is on a depth axis with respect to the focus adjusting device, in this x-y-position according to the scan strategy, wherein multiple different x-y-positions are iteratively measured with respectively subsequent scanning along the z-axis according to this scan strategy. In other words, multiple consecutive points can be scanned along the depth direction, thus for example the direction, which leads from the entry surface to the contact surface. Subsequently, the position can be adjusted in the plane and the depth direction in this new position can again be scanned. This can be performed for multiple x-y-positions in the plane until a sufficiently high number of intersections with the contact surface and/or the entry surface is determined.

In a further advantageous embodiment, it is provided that the focus adjusting device is adjusted for focusing the measurement laser radiation on a z-position, which is located in a depth axis situated parallel to the radiation direction of the focus adjusting device, and multiple different x-y-positions, which are located in an x-y-surface situated perpendicularly to the radiation direction of the focus adjusting device, are scanned in this z-position according to a pattern, a spiral and/or concentric circles according to the scan strategy, wherein multiple different z-positions are iteratively measured with respectively subsequent scanning of the x-y-surface according to this scan strategy. In other words, a plane in the depth direction is set in this embodiment, wherein this plane, which is spanned in the x-y-direction, is subsequently scanned. Herein, a pattern can be used, preferably one of the above-mentioned patterns or distributions, a spiral path from the inside to the outside or from the outside to the inside can be scanned in this surface and/or multiple concentric circles can be scanned to obtain intersections with the contact surface and/or entry surface. Subsequently, the position in the depth direction can be changed, which means that a next plane is accessed, in which this scan strategy is repeated. After accessing multiple different z-positions, thus, a space or volume can be scanned to obtain the intersections with the contact surface and/or entry surface.

Preferably, it is provided that one or more helix curves are scanned according to the scan strategy. This means that a measurement path can be provided for the measurement laser radiation, which scans a space in helical manner or as a helix, in which the contact surface and/or the entry surface are expected. After one-time scanning a helix curve, a radius of the helix curve can for example be changed and a new scan can occur. Thus, multiple different radii can for example be iteratively used, with which helix curves are scanned to scan the space, in which the contact surface and/or the entry surface are situated.

Preferably, it is provided that one or more planes situated obliquely with respect to the focus adjusting device are scanned according to the scan strategy. In other words, the focus adjusting device can use both the x-y control and the z control to scan a measurement plane situated obliquely in the space.

A further advantageous embodiment provides that a density of scan points in the vicinity of the intersection is increased after finding an intersection. In particular, the density of scan points in the vicinity of the intersection can be increased compared to focus points, at which an intersection is not determined. In other words, an approximate position of the respective surface can thus be determined by finding at least one intersection, wherein the scan around this intersection is subsequently refined, for example doubled, to obtain sufficient intersections for the adaptation of the three-dimensional surface model. By this embodiment, the advantage arises that the method can be accelerated.

Preferably, it can also be provided that if a basic shape of the contact element is known, the measurement laser radiation is adjusted along a surface to be expected from the basic shape, preferably with preset variances, to increase the number of the intersections. Thus, a determination of the shape of the surface can be accelerated and/or improved.

A further advantageous embodiment provides that the measurement laser radiation is provided from a laser radiation source also provided for generating the processing laser radiation. Thus, the material processing apparatus can preferably comprise only one laser, which can generate processing laser radiation and also provides measurement laser radiation for example by a reduced laser energy, in particular by an energy reducer. By this embodiment, the advantage arises that the use of a further laser for generating the measurement laser radiation can be omitted, which saves cost.

Particularly preferably, it is provided that the material processing apparatus is prepared for an eye laser treatment. In other words, the material processing apparatus can be a treatment apparatus for treating a human or animal eye, wherein a contact element for fixing the eye for the treatment is measured and the contact surface and/or entry surface thereof is determined by means of the method.

A further aspect of the invention relates to a material processing apparatus, in particular with at least one ophthalmological laser for the treatment of a human or animal eye, and a contact element fixable thereto. For example, the treatment of the eye can include a separation of a lenticule from a cornea with predefined interfaces by optical breakthroughs and/or an ablation of the cornea and/or a laser-induced refractive index change. Accordingly, the material processing apparatus can be formed to perform a method according to any one of the preceding embodiments.

In other words, the material processing apparatus can be formed as a treatment apparatus with at least one ophthalmological laser, at least one focus adjusting device or beam deflecting device and a fixing device, wherein a control unit of the material processing apparatus can for example be formed to perform a method according to any one of the preceding embodiments.

The control unit or the control device can for example be configured as a control chip, control unit or application program (“app”). The control device can preferably comprise a processor device and/or a data storage. By a processor device, an appliance or an appliance component for electronic data processing is understood. For example, the processor device can comprise at least one microcontroller and/or at least one microprocessor. Preferably, a program code for performing the method can be stored on the optional data storage. The program code can be configured, upon execution by a processor device, to cause the control device to perform one of the described embodiments of the method.

Preferably, the laser can be suitable to emit laser pulses in a wavelength range between 300 nanometers and 1400 nanometers, preferably between 700 nanometers and 1200 nanometers, at a respective pulse duration between one femtosecond and one nanosecond, preferably between ten femtoseconds and ten picoseconds, and a repetition frequency of greater than ten kilohertz, preferably between 100 kilohertz and 100 megahertz. Such a femtosecond laser is particularly suitable for producing volume bodies within the cornea.

Preferably, the material processing apparatus can comprise the control device with at least one storage device for at least temporary storage of at least one control dataset, wherein the control dataset or datasets can include control data for positioning and/or for focusing individual laser pulses into the cornea and/or the contact element.

Further features and the advantages thereof can be taken from the descriptions of the inventive aspects, wherein advantageous configurations of each inventive aspect are to be regarded as advantageous configurations of the respectively other inventive aspect.

A further aspect of the invention relates to a computer program including commands, which cause the material processing apparatus to execute method steps according to any one of the preceding embodiments.

According to the invention, a computer-readable medium is also provided, on which the computer program according to the preceding inventive aspect is stored. Herein, the same advantages and possibilities of variation as in the further inventive aspects arise.

### BRIEF DESCRIPTION OF THE FIGURES

Further features of the invention are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

FIG. 1 is a schematic representation of a material processing apparatus according to an exemplary embodiment.

FIG. 2 depicts schematic patterns for a scan strategy.

In the figures, identical or functionally identical elements are provided with the same reference characters.

### DETAILED DESCRIPTION

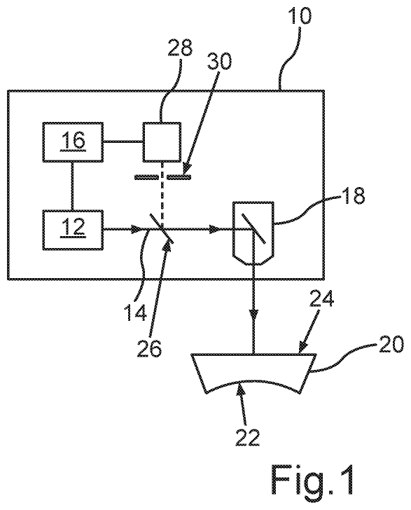

In FIG. 1, a severely schematized representation of a material processing apparatus 10, in particular of a treatment apparatus 10, for treatment of an eye is illustrated. The material processing apparatus 10 comprises a laser 12, which is formed to generate processing laser radiation, wherein the processing laser radiation can generate optical breakthroughs in an object (not shown) for processing the object. Furthermore, the laser 12 can be formed to generate measurement laser radiation 14, wherein the energy of the measurement laser radiation is below the energy for an optical breakthrough, in particular below 1 Joule per square centimeter. Preferably, the material processing apparatus 10 and the laser 12 can be provided for an eye laser treatment.

Besides the laser 12, the material processing apparatus 10 can comprise a control device 16, which can be formed to control the laser 12 by control data, such that it can emit pulsed laser pulses, for example for the treatment of an eye. Furthermore, the control device 16 can control a three-dimensionally acting focus adjusting device 18, such that the focus adjusting device 18 focuses the processing laser beam and/or the measurement laser beam 14 on preset positions, in particular positions preset by control data in or on the object and/or a contact element 20.

Preferably, the laser 12 can be a photodisruptive and/or ablative laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz. Optionally, the control device 16 additionally comprises a storage device (not illustrated) for at least temporary storage of at least one control dataset, wherein the control dataset or datasets include(s) control data for positioning and/or focusing individual laser pulses.

Further, the material processing apparatus 10 can include a contact element 20, which is transparent to the processing laser radiation. Preferably, the contact element 20 can be attached to the material processing apparatus 10. The contact element 20 can be provided to fix an object, for example an eye, in a position for the processing with the laser 12. Thereto, the contact element can comprise a contact surface 22, which represents a side of the contact element 20 to be fitted onto the object. Preferably, the contact surface 22 has a shape, which is adapted to the processing of the object, wherein a curved shape, for example a semi-circle, can be provided in case of an eye treatment. On the side facing the material processing apparatus 10, the contact element 20 can comprise an entry surface 24, through which the laser radiation penetrates the contact element 20 transparent to the processing laser radiation.

For preparing the material processing apparatus 10, the shape of the contact surface 22 and/or of the entry surface 24 can be determined after attaching the contact element. Hereto, the laser 12 can focus measurement laser radiation 14 on the contact surface 22 and/or the entry surface 24 by means of the focus adjusting device 18, wherein a reflection signal arises at a refractive index transition on the respective surface, which can be measured as an intersection with the contact surface 22 and/or the entry surface 24. This measurement of the backscattered and/or back-reflected radiation can preferably be confocally effected, wherein the backscattered or back-reflected radiation is radiated back into the material processing apparatus 10 through the focus adjusting device 18 in reverse direction of the optical path hereto, wherein this backscattered or back-reflected radiation can be measured by a detector 28 by a beam splitter 26. A pinhole 30 can be arranged in front of the detector 28 such that only that back-reflected radiation is measured, which originates from the focus point of the focus adjusting device. Since the principle of the confocal measurement is known, further details to optical components of the confocal measurement are not further described for reasons of clarity.

After detection of the backscattered or back-reflected radiation by the detector 28, the associated adjustment of the variable focus adjusting device 18 can be used to thus determine the position of intersections on the contact surface 22 and/or the entry surface 24.

When the position of the intersections on the contact surface 22 and/or the entry surface 24 of the contact element 20 is known, a three-dimensional surface model can be adapted or fitted thereto, for example by the control device 16, wherein the three-dimensional shape of the contact surface 22 and/or of the entry surface 24 is provided by the surface model.

For example, polygons, which together result in the three-dimensional shape, can be adapted to the determined intersections with the respective surface, which can be present in the form of a grid structure. Alternatively, or additionally, polynomials or a polynomial line (splines) can be adapted to the intersections of the contact surface 22 and/or entry surface 24, which together result in the shape of the respective surface. Zernike polynomials or Fourier series can also be particularly adapted to the intersections of the respective surface for determining the shape.

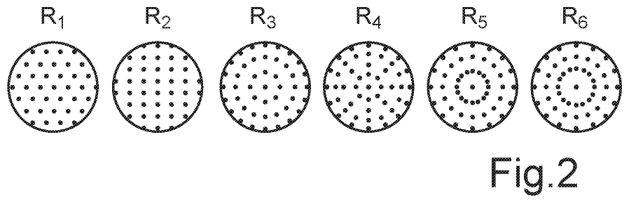

In order to obtain sufficient intersections with the contact element 20, it can additionally be preferably provided that a preset scan strategy is used, which focuses the measurement laser radiation 14 near or on the contact surface 22 and/or entry surface 24. A density can also be preset by the scan strategy, how many focus points are scanned in a spatial area, in which the contact surface 22 and/or the entry surface 24 are expected. Exemplary patterns or distributions, which can be used for the scan strategy, are illustrated in FIG. 2.

Thus, hexagonal distributions can for example be provided as the scan strategy, as illustrated in the pattern R₁ of FIG. 2. Alternatively, rectangular patterns can be used as the scan strategy, as shown in the pattern R₂. Further possibilities are circular patterns R₃, an Albrecht distribution R₄, a Jacobi distribution R₅ and/or a Legendre distribution R₆.

Particularly, an intersection with the contact surface 22 and/or entry surface 24 can also first be searched by the confocal measurement, wherein a density of scan points is increased in the area, in which the intersection has been found, to obtain an improved resolution of the respective surface. It can also be provided that the respective surfaces are scanned plane by plane, which means that an x-y-surface is scanned, and subsequently a z-position (depth direction) is changed, and the x-y-surface in this z-position is again measured. Herein, the patterns shown in FIG. 2 can for example be used and/or a spiral and/or concentric circles can be scanned. Instead of plane by plane in x-y-direction, scanning in respective z-direction can also be effected, wherein the x-y-position is subsequently shifted and the associated z-direction is scanned. Further scan strategies are helix curves, in particular with variable radius and/or planes situated obliquely to the focus adjusting device 18, which intersect the contact surface 22 and/or the entry surface 24.

Overall, the examples show, how the shape of the contact surface 22 and/or of the entry surface 24 can be determined without previous knowledge of the exact parameters of the contact element 20, to prepare the material processing apparatus 10 for processing an object.

1. A method for preparing a material processing apparatus for material processing by generating optical breakthroughs in or on an object, which comprises a variable, three-dimensionally acting focus adjusting device for focusing processing laser radiation on various locations in or on the object,
wherein a contact element to be fitted onto the object is attached to the material processing apparatus, wherein the contact element is transparent to the processing laser radiation, comprises a contact surface on its side to be fitted onto the object, and an entry surface for the processing laser radiation on its side facing the material processing apparatus,
wherein a shape of the contact surface and/or entry surface is determined by means of radiation of measurement laser radiation onto the contact surface and/or entry surface before the processing of the object, in that the measurement laser radiation is focused near or on the contact surface and/or entry surface by means of the variable focus adjusting device, wherein an energy density of the focused measurement laser radiation is too low for generating an optical breakthrough,
wherein radiation backscattered or back-reflected from the focus of the measurement laser radiation is confocally detected,
wherein a position of intersections on the contact surface and/or entry surface is determined from the confocally detected radiation and the associated adjustment of the variable focus adjusting device, and
wherein a three-dimensional surface model is adapted to the determined position of the intersections, wherein the three-dimensional shape of the contact surface and/or of the entry surface is provided by the surface model.

2. The method according to claim 1, wherein a grid structure is provided by the position of the intersections on the contact surface and/or the entry surface, and wherein polygons are adapted to the grid structure as the three-dimensional surface model.

3. The method according to claim 1, wherein polynomials, in particular a polynomial line, are adapted to the intersections on the contact surface and/or the entry surface.

4. The method according to claim 1, wherein Zernike polynomials or a Fourier series are adapted to the intersections on the contact surface and/or the entry surface as the three-dimensional surface model.

5. The method according to claim 1, wherein the measurement laser radiation is focused near or on the contact surface and/or the entry surface according to a preset scan strategy.

6. The method according to claim 5, wherein focus points of the measurement laser radiation are uniformly distributed in a spatial area, in which the contact surface and/or the entry surface are expected, according to the preset scan strategy.

7. The method according to claim 5, wherein the focus adjusting device is adjusted to an x-y-position for focusing the measurement laser radiation, which is located in a surface situated perpendicularly to the radiation direction of the focus adjusting device, and multiple focus points are scanned along a z-axis, which is located on a depth axis with respect to the focus adjusting device, in this x-y-position according to the preset scan strategy, wherein multiple different x-y-positions are iteratively measured with respectively subsequent scanning along the z-axis according to the preset scan strategy.

8. The method according to claim 5, wherein the focus adjusting device is adjusted to a z-position, which is located in a depth axis situated parallel to the radiation direction of the focus adjusting device for focusing the measurement laser radiation, and multiple different x-y-positions, which are located in an x-y-surface situated perpendicularly to the radiation direction of the focus adjusting device, are scanned in this z-position according to a pattern, a spiral and/or concentric circles according to the preset scan strategy, wherein multiple different z-positions are iteratively measured with respectively subsequent scanning of the x-y-surface according to the preset scan strategy.

9. The method according to claim 5, wherein one or more helix curves are scanned according to the preset scan strategy.

10. The method according to claim 5, wherein one or more planes situated obliquely with respect to the focus adjusting device are scanned according to the preset scan strategy.

11. The method according to claim 5, wherein after finding an intersection, a density of scan points in the vicinity of the intersection is increased.

12. The method according to claim 1, wherein the measurement laser radiation is provided from a laser radiation source also provided for generating the processing laser radiation.

13. The method according to claim 1, wherein the material processing apparatus is prepared for an eye laser treatment.

14. A material processing apparatus, in particular with at least one eye surgical laser for the treatment of a human or animal eye, and a contact element fixable thereto, wherein the material processing apparatus is formed to perform a method according to claim 1.

15. A computer program including commands, which cause a material processing apparatus, with at least one eye surgical laser for the treatment of a human or animal eye and a contact element fixable thereto, to execute the method steps according to claim 1.

16. A non-transitory computer-readable medium, on which the computer program according to claim 15 is stored.
